(19) United States
(12) Patent Application Publication
(10) Pub. No.: US 2007/0088229 A1
(43) Pub. Date: Apr. 19, 2007

(54) Needle assembly for a blood sampling device

(75) Inventor: Peter Jeffrey, Liverpool (GB)

Correspondence Address: CHAPMAN AND CUTLER, 111 WEST MONROE STREET, CHICAGO, IL, 60603, US

(73) Assignee: SAFE-T LIMITED, Lonan (GB)

(21) Appl. No.: 11/523,836

(22) Filed: Sep. 20, 2006

Related U.S. Application Data
(63) Continuation-in-part of application No. PCT/GB05/01061, filed on Mar. 22, 2005.

(30) Foreign Application Priority Data
Mar. 23, 2004 (GB) 0406438.2

(51) Int. Cl. A61B 5/00; A61M 5/32

(52) U.S. Cl. 600/576; 604/411

(57) ABSTRACT

First and second hollow needles extend from opposing sides of a common hub which is formed in one piece of transparent material. Proximal ends of the respective needles are in communication with each other by a narrow passageway through the hub. The projecting portion of the second needle is covered by an elastomeric sheath. The hub preferably includes a magnifying formation overlying the passageway portion between the proximal ends of the needles. In use, once the point of the first needle penetrates a vein, the patient's blood pressure is sufficient to displace air from the entire first needle and at least part of the passageway through the hub. The sheath over the second needle is sufficiently elastic to allow this and expand accordingly. The blood flows into the piercing needle and the passageway between the needles, thus being viewable through the surrounding transparent material and the magnifying portion, if present.

This is a continuation-in-part of International Application PCT/GB2005/001061, with an international filing date of Mar. 22, 2005. Priority is claimed to United Kingdom Application Serial No. GB 0406438.2, filed on Mar. 23, 2004, which is hereby incorporated by reference.

### TECHNICAL FIELD OF THE INVENTION

This invention concerns a needle assembly for a blood sampling device.

### BACKGROUND ART

Conventional blood samplers comprise a hollow needle mounted in and extending through a hub, by means of which the device may be held by a user and optionally mounted within a holder or the like. The needle is of stainless steel and has a sharp point at each end. One end is intended for insertion into a patient's vein. The other end is designed for insertion into a pre-evacuated collection tube, its sharp point being necessary to pierce a sealed septum of the collection tube. With the first end appropriately inserted into a vein, blood is sucked into the collection tube by vacuum as soon as the septum is pierced. Multiple blood samples may be taken successively from the same patient via the same needle into a succession of pre-evacuated collection tubes. When the required number of samples have been collected, the needle is withdrawn from the vein and the sampler is either discarded, or alternatively, if the sampler is mounted in a holder as described in our earlier patent specification EP-A-0648136, it is automatically retracted under spring bias into the syringe body.

There is one serious problem with conventional blood sampling devices. The user cannot be sure that the needle tip is appropriately located in a patient's vein until after the pre-evacuated collection tube has been applied to the other end and blood is seen to be entering the tube. If the needle tip is not in the vein, the vacuum will suck in tissue, which can be painful for the patient and lead to significant bruising. Also the collection tube, which is relatively expensive, will be wasted as its septum has been pierced and the vacuum at least partially lost. The patient has to suffer a further attempt to locate a vein and time is wasted.

Accordingly, it is a well established fact in the field of phlebotomy (blood sampling and analysis) that there are huge potential advantages during the early part of the sampling procedure if the presence of the needle tip (from where the blood is sucked into the sample tube) in a vein could be determined by the operator witnessing sight of some blood at an early stage of the procedure. A suitable location for such witnessing is in the hub of the multi-sampling needle where a gap may be created between the forward (skin penetrating) part of the needle and the distal (sample tube penetrating) part of the needle.

Many prior patents specifications have taught such a procedure but all have required that the air in the forward part of the needle must be vented and/or evacuated in order to allow the blood to pass up the needle to the witnessing chamber. Such arrangements are so expensive/complicated or otherwise impractical that none of the many earlier designs have gone into production.

For example, U.S. Pat. No. 4,679,571 discloses two separate needles mounted into a housing which provides a passageway between them and has a chamber with transparent walls through which the presence or absence of a blood sample will be visually apparent. The housing is formed of two parts which fit together so that one projects into the other and an annular space between the parts provides the chamber for viewing the presence of a sample. A relatively large initial volume of any sample is bled off into the annular chamber, and this can only be accomplished by venting air from the chamber. In order to visualise the sample in the chamber and also as a measure to seal off the chamber to prevent leaking a swellable tablet or sleeve of a proprietary starch graft co-polymer is located in the chamber.

U.S. Pat. No. 5,450,856 also discloses two needles mounted into a bulb of transparent plastics material which provides a blood witnessing chamber. Air is vented from the chamber by way of an aperture which is openable by depression of a button located therein. Additional or alternative venting of air is disclosed to be by way of an aperture in an elastomeric sheath over the needle which is intended for insertion, in use, into the evacuated collection tube.

U.S. Pat. No. 4,444,203 similarly discloses a transparent walled witnessing chamber in the hub which is vented by an aperture.

Current health regulations preclude any possibility of leakage from a device used for taking blood samples, so any device requiring venting from a visualisation or ‘flash’ chamber is impractical.

GB 2029228 discloses an alternative option in which there is still a witnessing passageway in the transparent hub between the ends of the respective needles which project therefrom. However, instead of venting from an outlet in the hub, a partial vacuum is applied to the end of the second needle after the first needle has been used to penetrate a blood vessel. This is done by providing a specially modified stopper to the collection tube and a special holder for fitting the hub thereto. The stopper has an intermediate cavity delimited by a diaphragm. By pushing the second needle into the stopper only so far as the intermediate cavity, a sufficient suction pressure is applied, by depression of the diaphragm towards the evacuated chamber of the collection tube, to draw air through the needles and draw blood at least to the witnessing passageway.

Other proposals have been made, but all involve complex multi-part assemblies which do not preclude leakage of the sampled fluid.

### OBJECTS OF THE INVENTION

An object of the invention is to overcome all the aforesaid problems and provide a cost-effective needle assembly for a body fluid sampling device which will, without any risk of leakage, provide a reliable visual indication that the needle tip is correctly located in a vein before a pre-evacuated collection tube is applied.

A more specific secondary object is to provide means, in such a needle assembly, to differentiate between blood coming from a true vein and that coming from a minor blood vessel.

### SUMMARY OF THE INVENTION

The main object is achieved by providing a needle assembly for a blood sampling device in which the transparent hub is formed in one piece without any means being provided in the hub for venting of the passageway between the ends of the respective needles. The passageway or duct between the proximal ends of the respective needles is kept as short and as narrow as is feasible. A patient's blood pressure should then be sufficient to force some blood into the blood vessel piercing needle and into the connecting duct between the needles, where it can be viewed through the surrounding transparent material. Air displaced from the passageway as blood flows in will pass out of the second needle and an elastomeric sheath thereover is sufficiently elastic/flexible to allow for this.

As needles conventionally used in blood sampling devices may have an outer diameter (OD) of about 0.9 to 1.0 mm, the passageway between the needle ends may be as little as 0.8 mm wide in many embodiments of the present invention. Only the end sections of the passageway where end regions of the needles are accommodated need to be equal to the OD of the needles. Thus, in accordance with the invention there is no intermediate chamber to which blood must pass in order to be observed and no requirement for venting such chamber. Indeed, there is no possibility of leakage because of the one piece hub formation.

The air in the first needle and in the witness chamber must be displaced in order to allow blood to pass up the needle and into the witness chamber so that it can be seen by the operative. If this air is simply vented to the atmosphere or evacuated by vacuum or other suction method, as in the prior art, there is no way that the operative can be sure that the needle point is in fact in a true vein and not in a minor blood vessel. This potential error can lead to the waste of the operative's time, much discomfort to the patient and a financial loss due the waste of only partially filled sample tubes which must be discarded.

It is known that the natural pressure of the blood inside the human body varies depending upon the nature of the vessel which contains the blood. The blood in a minor blood vessel is at a lower pressure than that in a true vein and also the flow rate and volume is much lower in the minor blood vessel than in the true vein. Using this knowledge it is possible to differentiate between the two.

Conventional multi-sample needles have an elastomeric sheath over the distal end of the needle to prevent the uncontrolled escape of blood between the application of a number of pre-evacuated (or piston evacuated) sample tubes. This sheath is pierced by the point of the distal end of the needle when the sample tube is applied and is pushed back down the needle by the septum of the sample tube. When the sample tube is removed, the sheath moves back over the needle point (due to its elasticity) and seals it, thus preventing the uncontrolled escape of blood under the natural blood pressure within the body of the patient.

To meet the secondary objective of the invention the elastic properties of the sheath are selected so that the sheath is able to expand only when blood from a true vein is applied to the end of the first needle and not when blood from a minor blood vessel is applied to the end of the first needle.

Because the sheath will only expand to accommodate displaced air when the flow from a vein is applied, blood will only be able to travel to the passageway in the hub for witnessing when the tip of the first needle has penetrated a true vein.

Advantageously at least part of the visible passageway between the proximal ends of the needles is magnified so that a user may more easily witness introduction of blood or other body fluid into said passageway. Thus, a magnifying formation acting as a magnifying lens preferably overlies at least part of the passageway.

Because this invention requires two separate needles, it permits another advantageous development, namely the displacement of one of the two needles out of alignment with the axis of the other of the two needles.

In practice, the needle intended to enter the patient's vein may be offset from the axis of the device, whereas the needle intended to enter the collection tube will need to remain in an axial disposition so as to pierce the septum of the collection tube centrally, at its weakest position, as the hub fits into the neck of the tube.

Such an offset disposition for the patient piercing needle has the advantage of allowing a lower angle of entry into the patient. This makes it easier to locate the needle tip into a vein without piercing the other (inner) wall of the vein, which is often a difficult task, particularly in very young, elderly or overweight patients.

Essentially, there are three possibilities for such an offset disposition. First, the first and second needles have principal axes that are parallel to one another. Second, the first and second needles have their proximal ends substantially in alignment but one of the needles extends at an oblique angle relative to the other so that the principal axes of the respective needles do not coincide. The third possibility is that the proximal ends of the first and second needles are offset as well as one needle extending at an oblique angle relative to the other. That angle in either the second or third possible arrangement may be from 135° to 179°. In other words the principal axes of the first and second hollow needles intersect at an angle of between 1° and 45°.

### BRIEF DESCRIPTION OF DRAWINGS

The invention will be described further, by way of example, with reference to the accompanying drawings, in which:

FIG. 1 is a fragmentary longitudinal cross-section of a first practical embodiment of a needle assembly according to the present invention when mounted into a syringe body with needle retraction provision;

FIG. 2 is a side elevation of a second practical embodiment of a needle assembly (sampler device) according to the invention;

FIG. 3 is a fragmentary, reduced scale partially sectional side view showing the device of FIG. 2 mounted into a syringe body with needle retraction provision in an “in use” position;

FIG. 4 is a side elevation, similar to FIG. 2, of a third practical embodiment of a needle assembly according to the present invention;

FIG. 5 is a side elevation, also similar to FIG. 2, of a fourth practical embodiment of a needle assembly according to the present invention;

FIG. 6 is an enlarged schematic view of the upper part of the needle assembly shown in FIG. 1;

FIG. 7 shows the same enlarged region as in FIG. 6 after the other end of the needle assembly has penetrated a vein; and

FIG. 8 is an enlarged scale transverse cross-section of a needle and its sheath in accordance with another embodiment of the present invention wherein the sheath is only partially thinned.

### DETAILED DESCRIPTION OF ILLUSTRATED EMBODIMENTS

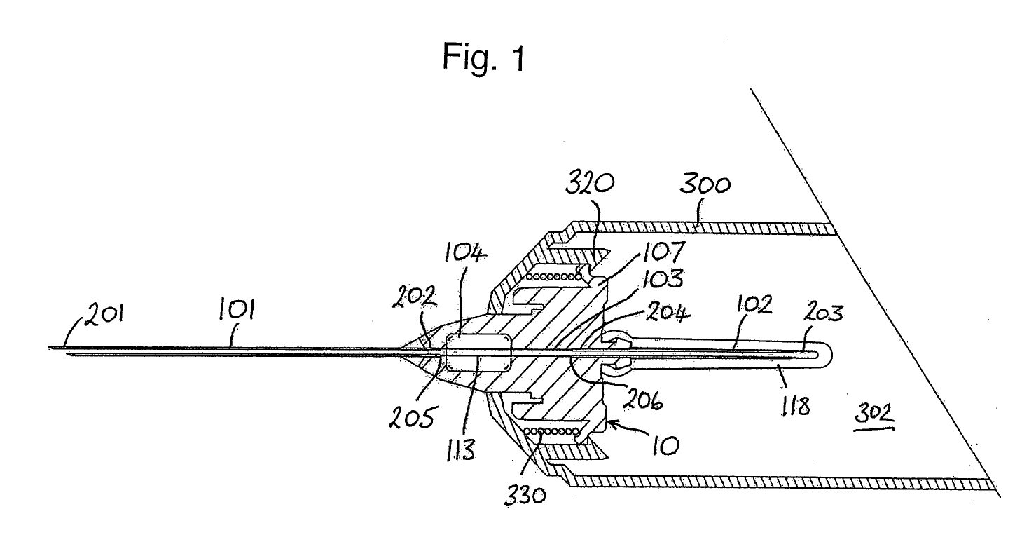

Referring to FIG. 1, a first embodiment of the invention is an in-line witnessing multi-sample needle assembly 10. It comprises a first hollow hypodermic needle 101, a second hollow hypodermic needle 102, a hub 107 and a flexible elastomeric sheath 118 over the second needle 102. The first and second needles 101, 102 extend from opposed ends of the hub 107 in alignment with each other, i.e. co-incident axes.

Both of the needles 101, 102 are necessarily of stainless steel or comparable material to meet required standards for medical devices.

The first needle 101 has, at one end, a multi-ground point 201 for penetration of a patients' skin and vein wall, while its other end 202 is plain and is bonded to the hub 107. The second needle 102 also has a ground point 203 at its free end, which is for piercing of a septum of a pre-evacuated blood sampling tube (not shown). The elastomeric sheath 118 would also be pierced in that operation. The other end 204 of the second needle 102 is also plain and is also bonded to the hub 107.

The hub 107 is formed in one piece of crystal clear (i.e. transparent) thermoplastic resin, such as a polycarbonate or ABS resin. A linear passageway 103 is formed through the hub 107. The end regions at the plain ends 202, 204 of the respective needles 101, 201 extend a short distance into opposing wider end sections of the passageway 103 up to respective locating shoulders 205, 206 which limit the insertion of the respective needle ends 202, 204 during manufacture of the device. The respective end regions of the needles 101, 201 are securely bonded in the end sections of the passageway 103 by any suitable adhesive.

A gap remains between the shoulders 205, 206 and therefore between the needle ends 202, 204. This gap provided by a central section 113 of the passageway 103, enables fluid, particularly blood, flowing into the passageway 103 to be seen by a user of the device through the transparent hub material.

A convex magnifying formation 104 is provided as an integral part of the exterior surface of the hub 107 overlying the central section 113 of the passageway 103 between the ends 202, 204 of the needles 101, 102. This magnifying formation acts as a lens and enlarges the view of this passageway section 113. In practise, the convex formation 104 may be sunk into a recess in the exterior of the hub 107 so that the hub exterior remains generally symmetrical and is still able to fit with minimum clearance into a holder, such as a syringe body 300.

The passageway 103 through the hub 107 needs, in its end sections, to match the OD of the needles 101, 102, which is typically 0.9 to 1.0 mm in order to accommodate the end regions of same. The central section 113 of the passageway 103, between the shoulders 205, 206, which delimit the position of the needles, is narrower and may be about 0.8 mm.

In use, when the end 201 of the first needle 101 is correctly inserted into a vein of a patient, his/her blood pressure will be sufficient to force blood through the first needle 101 and into the central section 113 of the passageway 103. This blood will be visible in the central section 113 of the passageway 103, as viewed via the magnifying formation 104. Air displaced by the blood coming up into the passageway 103 will pass out of the end 203 of the second needle 102 and the sheath 118 is sufficiently elastic/flexible to accommodate the small volume of air expelled.

The sampler assembly 10 is shown mounted into a syringe body 300 of known type which has a single interior chamber 302 and latching provisions 320 in a front end of this chamber 302 for retaining the hub 107 against the bias of a spring 330. These latching provisions 320 are deflectable outwards e.g. by insertion of a hollow plunger (not shown) into the body 300 to release the spring 330 and retract the needle assembly 10, including the full extent of the first needle 101 into the chamber 302 and into the plunger, in a manner disclosed in the applicant's earlier EP-A-0648136.

The configuration of the hub 107 and the size of the opening in the front end of the syringe body 300 are such, in this embodiment, that a portion of the hub 107, including at least a portion of the magnifying formation 104, extend from the front end of the syringe body 300. This is to ensure clear visibility of the passageway 113, and witnessing of entry of blood when a vein is pierced, via the transparent hub material and the magnifying formation 104. Although the syringe body 300 will generally be formed of transparent material, such as transparent polypropylene, the visibility of the passageway section 113 may nevertheless be somewhat blurred if it has to be viewed through both the body 300 and the hub 107. Projection of part of the hub 107 out of the body 300 in this design avoids this potential problem. However, in other embodiments, substantially all of the hub 107, including all of the magnifier formation 104, may be located inside a transparent syringe body 300 with acceptable visibility of the witnessing duct 113.

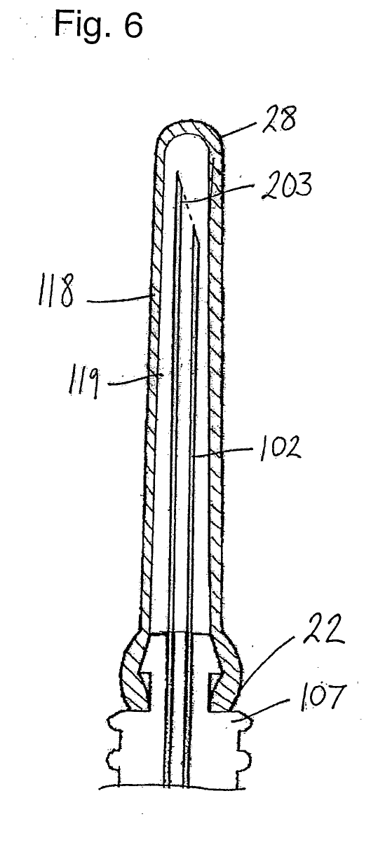

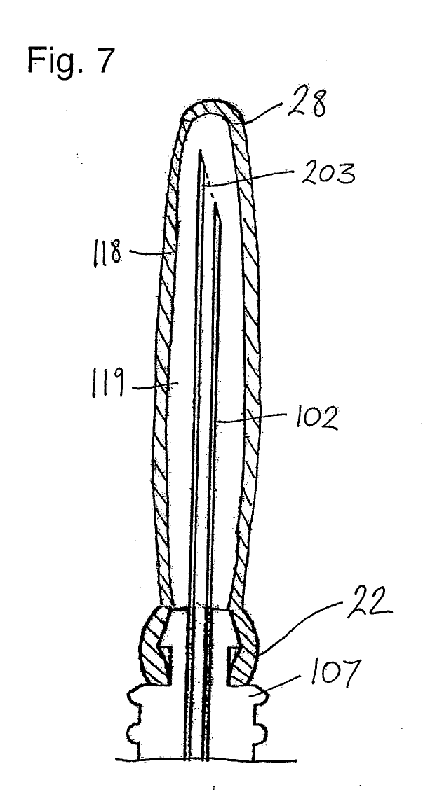

Referring now to FIGS. 6 and 7, the elastomeric sheath 118 initially has a closed end 28 and an open end 22, and its open end 22 fits sealingly onto the hub 107 adjacent the location where the second needle 102 emerges. A suitable material for the sheath is medical grade thermoplastic elastomer.

At least in part, the sheath 118 is more elastic, i.e. with a lower elastic modulus, than known such sheaths. Moreover, the sheath 118 has a thinner wall and a greater space 119 remaining between it and the needle 102 it covers compared to conventional such sheaths. By having such a thinner wall, the sheath 118 is able to expand under internal pressure compared to previously available needle sheaths. However, the properties, including the thickness, of the sheath 118 are chosen so that it only expands and allows blood to flow up to the witnessing passageway 113 of the transparent hub 107 when the first needle 101 has penetrated a vein. Thus, it is only the force/pressure of blood from a vein which will expand the sheath 118, and in the event that only a minor blood vessel is penetrated its force/pressure will not bring this about and no blood we'll be witnessed, making it apparent that the procedure has failed.

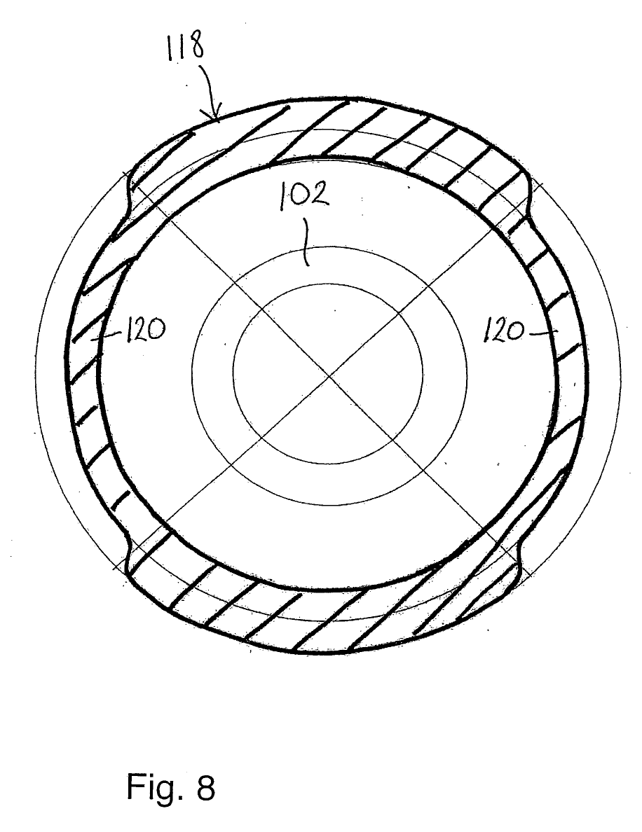

Although an enlarged annular space 119 is shown between the entire needle 102 and the sheath 118 in FIGS. 6 and 7, that would not necessarily always be the case. In other embodiments, as shown in FIG. 8, only parts of the wall are thinned in this way. Thus, as shown respectively in FIG. 8, only two alternate and symmetrically located longitudinal strips 120 of the wall are thinned in this way. In further embodiments a greater number of alternate longitudinal strips (as 120) may be provided, e.g. four or eight or more. A suitable thickness for at least one region of the sheath is less than 0.15 mm. Thus, either the entire sheath or specific regions such as the said longitudinal thinner strips 120 may have a thickness of less than 0.15 mm.

In use, as shown in FIG. 7, after the needle point 201 has penetrated a vein, blood pressure will be sufficient to displace air from the entire first needle 101 and at least part of the passageway 103 through the hub 107, thus expanding the sheath 118.

By witnessing the blood appearing inside the transparent hub 107, the operator can know that the needle point 201 has correctly penetrated a vein. Once that is known, the sheath covered end 203 of the needle 102 can be inserted into an evacuated collection tube. The end 203 then pierces both the sheath 118 and the septum of the collection tube in conventional manner.

The expansion of the sheath 118 has been exaggerated in FIG. 7 for purposes of illustration. Typically, the initial volume of the interior of the sheath is about 0.063 cm³ while the volume of the interior of the first needle 102 together with the interior of the hub passageway witnessing region 113 is about 0.005 cm³. Thus, the sheath 118 needs to expand only by about 8% of its volume to accommodate all the air displaced from the needle 102 and the witnessing passage 113. In order to take account of this the sheath 118 should preferably be able to expand sufficiently to increase its enclosed volume by up to about 15% of its enclosed volume.

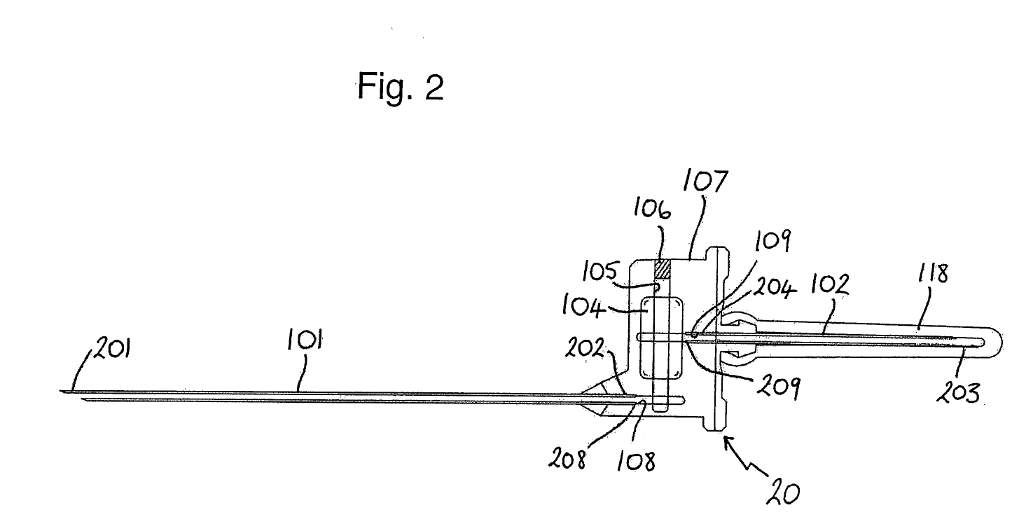

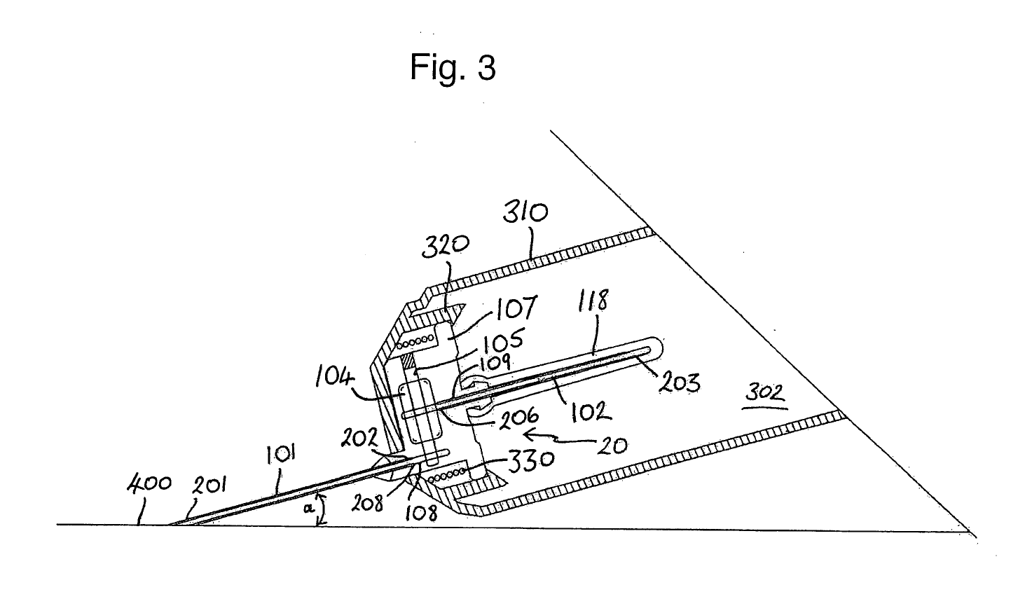

Referring now to FIGS. 2 and 3 a second embodiment of the invention is an offset witnessing multi-sample needle assembly 20. The same reference numerals are used to designate parts which correspond to those of the first embodiment and these will not be described again.

The proximal ends 202, 204 of the needles 101, 102 are in this embodiment located and bonded into axially offset portions 108, 109 of the passageway through the transparent hub 107. The needles 101, 102 have principle axes extending parallel to one other. As best shown in FIG. 3 the second needle 102 is still axial of the device and the syringe body 310 in which it can be mounted, whereas the first needle 101 is now offset from that axis.

The passageway through the hub 107 consists of the aforesaid axially offset portions 108, 109, which lie substantially parallel to each other but extend in from opposing sides of the hub, and a transverse bridging portion 105 which connects these two offset portions 108, 109. The offset portions 108, 109 of the passageway are formed as blind bores from opposing sides of the hub 107. Each has a narrower inner end region delimited by a respective shoulder 208, 209 which serves to limit insertion of the proximal ends 202, 204 of the respective needles 101, 102 during manufacture. The needles 101, 102 are bonded to the hub 107 once inserted. The bridging portion 105 of the passageway extends between and substantially perpendicular to the narrower inner end regions of the offset portions 108, 109 of the passageway.

The hub 107 is manufactured in one piece by injection moulding and the bridging portion 105 is formed during moulding by use of a sliding core. When the core is withdrawn the bridging portion 105 initially extends to, i.e. is open to, the outside of the hub 107. The open end may be sealed off by a plug 106, which may suitably be formed of polypropylene and may be a simple push fit therein. However, a more reliable leak proof closure to the bridging portion 105 may be achieved by shaping the mould for the hub 107 to provide additional material encircling the point of entry and exit of the sliding core. After the bridging portion 105 has been formed its open end is then closed off by a tool which softens the surrounding mound of material and pushes it into the open end.

Again the outer sections of the passageway through the hub 107 need to match the OD of the needles 101, 102 which they accommodate. The narrower inner ends of the blind bores 108, 109 may be of a diameter as little as 0.8 mm. The bridging portion 105 of the passageway may need to be of slightly larger diameter, such as 1.0 mm to 1.5 mm, owing to manufacturing constraints.

By use of this axially offset arrangement of needles the angle of attack (a) at the skin 400 of a patient (FIG. 3) can be reduced from a minimum of 16° as for existing single needle samplers or the in-line sampler 10 to a minimum of 4°.

Obviously the syringe body 310 must be appropriately provided with a non-axial opening at a suitable position for projection therethrough of the needle 101 and at least a small portion of the hub 107 where the passageway portion 108 is formed.

In other respects, this embodiment is used in exactly the same way as described for the first embodiment of FIG. 1.

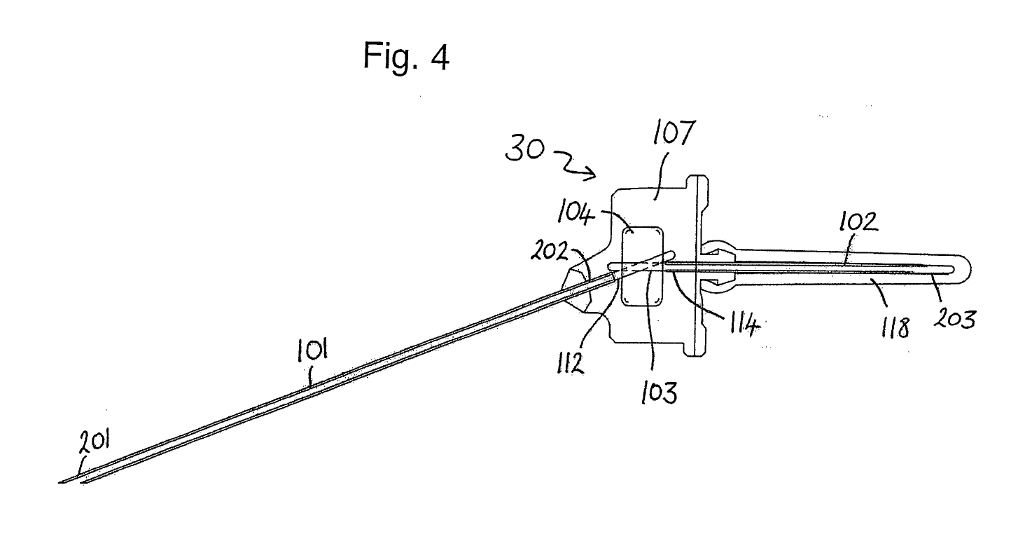

FIG. 4 shows a third embodiment 30 of the device of the invention which is akin to the first embodiment (FIG. 1). The same reference numerals are used for corresponding parts and these need not be described again. It differs from the first embodiment in that instead of a single linear passageway through the hub the passageway 103 is formed by respective blind bores 112, 114 which intersect at an angle of between 1° and 45°, preferably about 20° to 30°. In this respect, the bore 114, into which the needle 102 for insertion into the collection vessel is located, is axial, while the other bore 112, into which the needle 101 for piercing the vein is located, extends at an angle thereto. Thus, the through passageway 103 itself bends at an obtuse angle of between 135° and 179° (preferably 150° and 160°) and there are two blind ends of short extent. The intersection of the bores 112, 114 is generally in the middle of the hub 107 and is overlain by a magnifying formation 104 for witnessing of blood entry into the needle 101 as before.

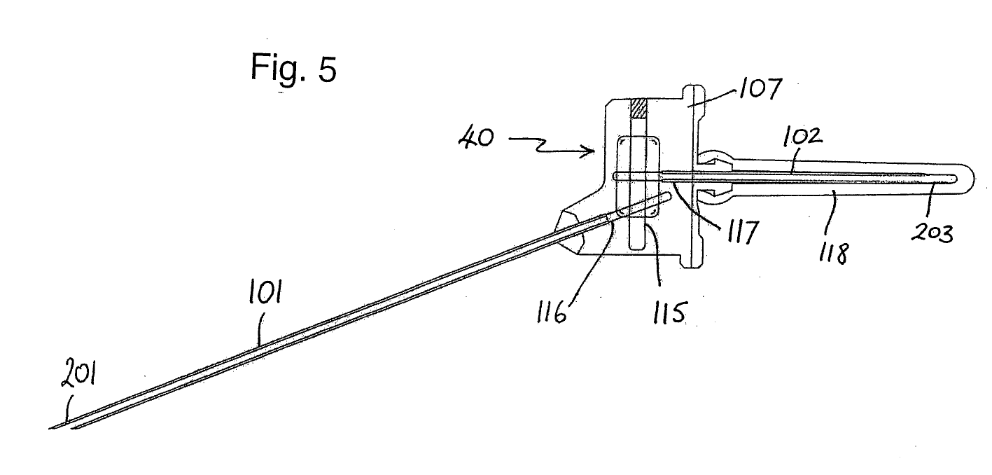

FIG. 5 shows a fourth embodiment 40 which bears resemblance to the embodiment of FIGS. 2 and 3 in that the passageway through the hub 107 is formed by two blind bores 116, 117 extending from opposing sides of the hub and connected by a transverse bridging portion 115. However, it is also similar to the preceding embodiment in that the blind bore 117 is axial of the hub 107, while the blind bore 116 is both offset by a short spacing and also extends at an angle of between 1° and 45° (preferably 20° and 30°) to the axis of the hub 107.

Both the aforesaid embodiments (FIGS. 4 and 5), in use, enable a reduced angle of attack at the skin of a patient.

The foregoing is illustrative not limitative of the scope of the invention. Many variations in detail are possible in other embodiments.

1. A needle assembly for a blood sampling device comprising first and second hollow needles extending from opposing sides of a common hub, proximal ends of the respective needles being in communication with each other by a passageway through the hub which is not provided with any vent to the exterior, the said common hub being formed in one piece of transparent material such that the passageway is visible from the exterior, in which respect the first needle has a pointed distal end suitable for penetrating a vein of a patient and the second needle has a pointed distal end suitable to pierce a septum of a conventional pre-evacuated blood sampling tube, and an elastomeric sheath which is mounted over the second needle so as to prevent escape of any fluid, but is sufficiently elastic to accommodate a volume of air expelled from the second needle when the free end of the first needle penetrates a vein.

2. A needle assembly as set forth in claim 1 wherein the elastomeric sheath has elastic properties selected so that the sheath is able to expand only when pressure from a true vein is applied to the end of the first needle and not when a lower pressure, as from a minor blood vessel, is applied to the end of the first needle.

3. A needle assembly as set forth in claim 1 wherein the thickness of the material of the sheath, at least in one region of the sheath, is less than 0.15 mm.

4. A needle assembly as set forth in claim 1 wherein the material of the sheath is sufficiently elastic to expand to accommodate an increase of up to 15% in its enclosed volume.

5. A needle assembly as set forth in claim 1 wherein the needles are linear and have principle axes that do not coincide.

6. A needle assembly as set forth in claim 5 wherein the passageway has respective axially offset sections, into which the proximal end regions of the first and second needles are mounted, and a bridging section which extends transversely between these axially offset sections.

7. A needle assembly as set forth in claim 6 wherein the bridging section extends beyond at least one of the axially offset sections of the passageway towards the outside of the hub where it is closed off.

8. A needle assembly as set forth in claim 1 wherein the hub includes a magnifying formation overlying the passageway between the proximal ends of the needles.

9. A blood sampling device comprising a substantially tubular holder body in combination with a needle assembly according to claim 8, in which respect the holder body has an interior chamber for reception of the collection tube and the hub of the needle assembly is located in a front end of said interior chamber of the holder body such that a portion of the hub including at least a portion of the magnifying formation as well as the first hollow needle extends from the front end of the body while the second hollow needle extends into the interior chamber thereof.

10. A needle assembly for a blood sampling device comprising first and second hollow needles extending from opposing sides of a common hub, proximal ends of the respective needles being in communication with each other by a passageway through the hub which is not provided with any vent to the exterior, the said common hub being formed in one piece of transparent material such that the passageway is visible from the exterior in which respect the first needle has a pointed distal end suitable for penetrating a vein of a patient, the second needle has a pointed distal end suitable to pierce a septum of a conventional pre-evacuated blood sampling tube, and the needles are linear and have principle axes that do not coincide.

11. A needle assembly as set forth in claim 10 wherein the needles are parallel to one another.

12. A needle assembly as set forth in claim 10 wherein the proximal ends of the respective needles are offset from each other.

13. A needle assembly as set forth in claim 10 wherein the principal axes of the first and second hollow needles intersect at an angle of between 1° and 45°.

14. A needle assembly as set forth in claim 10 wherein the passageway has respective axially offset sections, into which the proximal end regions of the first and second needles are mounted, and a bridging section which extends transversely between these axially offset sections.

15. A needle assembly as set forth in claim 14 wherein the bridging section extends beyond at least one of the axially offset sections of the passageway towards the outside of the hub where it is closed off.

16. A needle assembly as set forth in claim 10 wherein the hub includes a magnifying formation overlying the passageway between the proximal ends of the needles.
